(19) United States
(12) Patent Application Publication
(10) Pub. No.: US 2016/0158368 A1
(43) Pub. Date: Jun. 9, 2016

(54) METHODS AND COMPOSITIONS FOR THE TREATMENT OF HISTIOCYTOSIS

(71) Applicant: Therapure Biopharma Inc., Mississauga (CA)

(72) Inventors: Irina Ayres, Midland (CA); Murray J. Cutler, Markham (CA); Kathryn Matthews, Toronto (CA); Steven Brookes, Burlington (CA); J. Gordon Adamson, Georgetown (CA); David N. Bell, Oakville (CA)

(21) Appl. No.: 14/928,528

(22) Filed: Oct. 30, 2015

Related U.S. Application Data
(60) Provisional application No. 62/073,178, filed on Oct. 31, 2014.
(60) Provisional application No. 62/199,529, filed on Jul. 31, 2015.

(51) Int. Cl. A61K 47/48; A61K 31/7064; A61K 31/7076

(52) U.S. Cl. CPC A61K 47/48007; A61K 31/7076; A61K 31/7064; A61K 47/484

(57) ABSTRACT

The present application provides methods of treating CD163⁺ histiocytosis comprising administering an effective amount of a CD163 ligand-drug conjugate to an animal or cell in need thereof.

### CROSS REFERNCE TO RELATED APPLICATIONS

This application claims benefit under 35 U.S.C. §119(e) to United States Provisional Patent Application Ser. No. 62/073,178 (pending), filed on Oct. 31, 2014 and U.S. patent application Ser. No. 62/199,529 (pending), filed on Jul. 31, 2015, which are both incorporated herein by reference in their entirety.

### FIELD OF THE INVENTION

The present application relates to the use of drugs targeted to cells expressing CD163 for treating non-Langerhans forms of histiocytosis such as Erdheim-Chester disease (ECD). In particular, the application relates to the use of CD163 ligand-drug conjugates for treating CD163⁺ histiocytosis.

### BACKGROUND OF THE INVENTION

Erdheim-Chester disease is a rare, non-Langerhans form of histiocytosis. It is characterized by the xanthogranulomatous infiltration of tissues by histiocytes and the development of fibrosis.

Erdheim-Chester disease can be distinguished from Langerhans Cell Histocytosis (LCH) based on the immunological characteristics of the histiocytes; ECD histiocytes stain positively for CD68 and not C1a. Recently, several reports (Dickson et al, 2008; Gong et al 2009, Murakami et al 2011 and Purgina et al, 2011) have indicated that the histiocytes in patients with ECD also stain positively for CD163, the hemoglobin-haptoglobin receptor.

The hemoglobin-haptoglobin scavenger receptor, CD163, has been described on monocytes and macrophages (Kristiansen, 2001). This receptor scavenges the hemoglobin-haptoglobin complex by mediating endocytosis of the complex. CD163 belongs to the Group B scavenger receptor cysteine-rich superfamily, a family of receptors that includes CD5, CD6 and WC1. Previous studies of antibody-mediated crosslinking of CD163 on monocytes have demonstrated that ligation of the CD163 induces tyrosine kinase-dependant signaling resulting in the mobilization of intracellular calcium ions, inositol triphosphate production and increased secretion of anti-inflammatory cytokines (van den Heuvel et al, 1999).

The pathogenesis of ECD is unclear. However, recent observations indicate that ECD can be clonally driven and immune-mediated. ECD histiocytes show intense expression of chemokines and pro-inflammatory cytokines, such as tumor necrosis factor alpha (TNF-α) and interleukin 6 (IL-6) (Stoppacciaro et al., 2006; Dagna et al., 2010).

The first-line therapy for ECD is interferon alpha (IFNα) with variable efficacy and limited tolerance. Other therapies include anti-neoplastic agents such as vemurafenib, sirolimus, or imatinib. However, patients must take these drugs for the rest of their lives, and the therapies are associated with severe side effects, such as squamous cell carcinoma, skin rash (vemurafenib), and immune suppression (sirolimus and imatinib).

In view of the foregoing, there is a need in the art to develop improved methods for treating patients with non-Langerhans forms of histiocytosis.

### SUMMARY OF THE INVENTION

Histiocytes from patients with Erdheim Chester disease (ECD), a non-Langerhans form of histiocytosis, stain positively for CD163. Therefore, targeting a drug to CD163 on the histiocytes provides a means for treating ECD.

Accordingly, one aspect of the present application relates to a method for treating CD163⁺ histiocytosis comprising of administering an effective amount of a CD163 ligand-drug conjugate to an animal or cell in need thereof.

Also included is use of effective amount of a CD163 ligand-drug conjugate to an animal or cell in need thereof for treating CD163⁺ histiocytosis. Further provided is use of a CD163 ligand-drug conjugate in the preparation of a medicament for treating CD163⁺ histiocytosis in an animal or cell in need thereof. Even further provided is a CD163 ligand-drug conjugate for use in treating CD163⁺ histiocytosis in an animal or cell in need thereof.

In one embodiment, CD163⁺ histiocytosis is a non-Langerhans form of histiocytosis. In another embodiment, CD163⁺ histiocytosis is Erdheim-Chester disease.

The drug of the CD163 ligand-drug conjugate is optionally any anti-proliferative drug or any drug with immunomodulatory attributes. In one embodiment, the drug is cladribine. In another embodiment, the drug is ribavirin.

In another embodiment, the CD163 ligand is hemoglobin.

In another embodiment, the CD163 ligand is hemoglobin and the drug is cladribine. In another embodiment, the CD163 ligand is hemoglobin and the drug is ribavirin. In a further embodiment, the ligand-drug conjugate is hemoglobin-ribavirin in combination with hemoglobin-cladribine.

In another embodiment, the CD163 ligand is an anti-CD163 antibody conjugated to a drug such as cladribine or ribavirin. In another embodiment, the anti-CD163 antibody is conjugated to two drugs such as cladribine and ribavirin.

In a further embodiment, the animal is a human.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will now be described in relation to FIG. 1 to FIG. 4.

FIG. 1 describes the relationship between CD163⁺ histiocytes and hemoglobin-conjugated therapeutics such as hemoglobin conjugated cladribine and ribavirin.

FIG. 2 demonstrates the characterization of in vitro models of histiocytosis by analyzing expression of CD163, CD68, CD1a and s100 by flow cytometry.

FIG. 3 demonstrates the potency of hemoglobin-cladribine on in vitro models of histiocytosis.

FIG. 4 demonstrates apoptosis-inducing effect of hemoglobin-cladribine in an in vitro model of histiocytosis.

### DETAILED DESCRIPTION OF THE INVENTION

The inventors have developed a hemoglobin-cladribine drug conjugate based on the chemical conjugation of human hemoglobin to the drug cladribine. This drug conjugate allows for the targeting of cladribine to cells that express CD163 including the infiltrating histiocytes which characterize Erdheim-Chester disease, a non-Langerhans form of histiocytosis.

Accordingly, the application discloses methods for treating CD163⁺ histiocytosis such as Erdheim-Chester disease comprising administering an effective amount of a CD163 ligand-drug conjugate to an animal or cell in need thereof. Also disclosed is use of effective amount of a CD163 ligand-drug conjugate to an animal or cell in need thereof for treating CD163⁺ histiocytosis. Further disclosed is use of a CD163 ligand-drug conjugate in the preparation of a medicament for treating CD163⁺ histiocytosis in an animal or cell in need thereof. Even further disclosed is a CD163 ligand-drug conjugate for use in treating CD163⁺ histiocytosis in an animal or cell in need thereof.

The term “histiocytosis” refers to a group of syndromes that involve an abnormal increase in the number of histiocytes. Histiocytes are macrophages found in connective tissue. Types of histiocytosis include, but are not limited to, Langerhans Cell Histiocytosis, malignant histiocytosis syndrome and Non-Langerhans Cell Histiocytosis. Examples of Non-Langerhans cell histiocytosis inlcude Erdheim-Chester disease, Rosai-Dorfman disease, histiocytic sarcomas or hemophagocytic lymphohistiocytosis.

The phrase “CD163⁺ histiocytosis” refers to any form of histiocytosis that is characterized by histiocytes that express the hemoglobin-haptoglobin scavenger receptor, CD163 or a CD163 related receptor. In one embodiment, at least 5, 10, 25, 50, 75, 85, 95 or 100% of the histiocytes in a patient with CD163⁺ histiocytosis express the hemoglobin-haptoglobin scavenger receptor, CD163 or a CD163 related receptor. In another embodiment, at least 5, 10, 25, 50, 75, 85, 95 or 100% of the histiocytes in a patient with CD163⁺ histiocytosis stain positively for the hemoglobin-haptoglobin scavenger receptor, CD163 or a CD163 related receptor (as in some cases reported in the literature, all histiocytes in the infiltrate stain positive for CD163). In one embodiment, CD163⁺ histiocytosis are found in patients with hemophagocytic lymphocytosis. In another embodiment, CD163⁺ histiocytes are found in patients with a non-Langherhans form of histiocytosis such as Erdheim-Chester disease, Rosai-Dorfman disease, histiocytic sarcomas or hemophagocytic lymphohistiocytosis.

Erdheim-Chester disease (ECD) is a rare, non-Langerhans form of histiocytosis that can be characterized by the xanthogranulomatous infiltration of tissues by histiocytes and the development of fibrosis. Histiocytes in patients with ECD also stain positively for the hemoglobin-haptoglobin scavenger receptor, CD163 (Dickson et al, 2008; Gong et al 2009, Murakami et al 2011 and Purgina et al, 2011). Accordingly, Erdheim-Chester disease is a CD163⁺ histiocytosis.

The term “CD163” as used herein means the hemoglobin-haptoglobin scavenger receptor that is expressed on macrophages, monocytes and CD34⁺ stem cells. It is a member of the Group B scavenger receptor cysteine-rich (SRCR) superfamily which may have the sequence as described in Law et al. (1993).

The phrase “CD163 ligand” refers to any substance that binds directly or indirectly to the hemoglobin-haptoglobin scavenger receptor, CD163 or a CD163 related receptor.

In one embodiment, the CD163 ligand is hemoglobin. In another embodiment, the CD163 ligand is a CD163 binding protein such as an antibody or antibody fragment that binds CD163.

The term “antibody” as used herein is intended to include monoclonal antibodies, polyclonal antibodies, and chimeric antibodies. The antibody may be from recombinant sources and/or produced in transgenic animals. The term “antibody fragment” as used herein is intended to include without limitations Fab, Fab′, F(ab′)₂, scFv, dsFv, ds-scFv, dimers, minibodies, diabodies, and multimers thereof, multispecific antibody fragments and Domain Antibodies.

The term “drug” as used herein refers to any therapeutically effective substance (i.e., substances that are capable of having a biological effect and/or altering the physiology of a patient).

The term “CD163 ligand-drug conjugate” as used herein refers to a drug that is bound directly or indirectly to a CD163 ligand. A drug may be conjugated or coupled to a ligand by any method known in the art. For example, a drug may be covalently linked directly to a ligand or a drug may be joined to a ligand through a linking agent, or linker.

Examples of CD163 ligand-drug conjugates include hemoglobin-cladribine and hemoglobin-ribavirin. In such conjugates, hemoglobin (a CD163 ligand) is chemically conjugated to cladribine or ribavirin, for example through the methods described by Brookes et al. (2006).

The CD163 ligand-drug conjugates described herein are effective for treating CD163⁺ histiocytosis such as Erdheim-Chester disease. In one embodiment, the drug of the ligand-drug conjugate is any drug that treats CD163⁺ histiocytosis when targeted to CD163 expressing histiocytes. In another embodiment, the drug is an anti-viral or immunomodulatory drug. Another example would include anti-proliferative drugs including nucleoside analogs or kinase inhibitors.

An anti-proliferative drug particularly useful in the ligand-drug conjugates described herein is cladribine. Cladribine (also known as 5-(6-amino-2-chloro-purin-9-yl)-2-(hydroxymethyl)oxolan-3-ol) is a drug used to treat hairy-cell leukemia and multiple sclerosis. Cladribine is a prodrug which when metabolized resembles purine DNA nucleosides, and as such inhibits adenosine deaminase and interferes with the DNA replication process. Cladribine has been demonstrated to be effective in the treatment of ECD. Further, as demonstated in the Examples, hemoglobin-cladribine conjugates are cytotoxic towards CD163⁺ histiocytes. Accordingly, hemoglobin-cladribine conjugates may be useful in treating CD163⁺ histiocytosis such as ECD. Conjugation to hemoglobin and targeting cladribine to CD163+ histiocytes avoids systemic distribution of the drug to non-target cells thus potentially allowing for an increase in safety (reduced systemic toxicity) and in potency through concentration in/to the target cells.

An anti-viral and immunomodulatory drug that may be useful in the ligand-drug conjugates described herein is the ribonucleoside ribavirin. Ribavirin (also known as 1-[(2R,3R,4S,5R)-3,4-dihydroxy-5-(hydroxymethyl)oxolan-2-yl]-1H-1,2,4-triazole-3-carboxamide) is a drug used to treat viral infections. Ribavirin is a prodrug which when metabolized resembles purine RNA nucleosides. It has been shown to have immunomodulatory attributes in treatment of chronic hepatitis C virus infection (Soota and Maliakkal, 2014). Ribavirin has been demonstrated to have immunomodulatory properties when conjugated to hemoglobin. For example, in a mouse model of viral hepatitis, hemoglobin-ribavirin was able to inhibit the production of pro-inflammatory cytokines interferon gamma (IFN-γ) and TNF-α by macrophages both in vitro and in vivo when compared to ribavirin or hemoglobin only after infection with the mouse hepatitis strain MHV-3 (Levy et al., 2006). In the study mice treated with hemoglobin-ribavirin had improved overall survival and clinical behaviour supporting the concept of improved efficacy through targeting to CD163+ cells while minimizing systemic distribution to non-target cells. In addition, in a mouse model of hemophagocytic lymphohistiocytosis, hemoglobin-ribavirin was able to modulate the cytokine profile of activated CD163⁺ macrophages and dramatically improve the course of the disease. Mice that were administered hemoglobin-ribavirin remained alive at the end of the study period (30 days) while those that did not receive hemoglobin-ribavirin did not survive beyond 20 days. In addition, levels of IFN-γ, a pro-inflammatory cytokine were reduced significantly in those animals administered hemoglobin-ribavirin. Accordingly, ribavirin-hemoglobin conjugates may be useful in the therapeutic methods described herein.

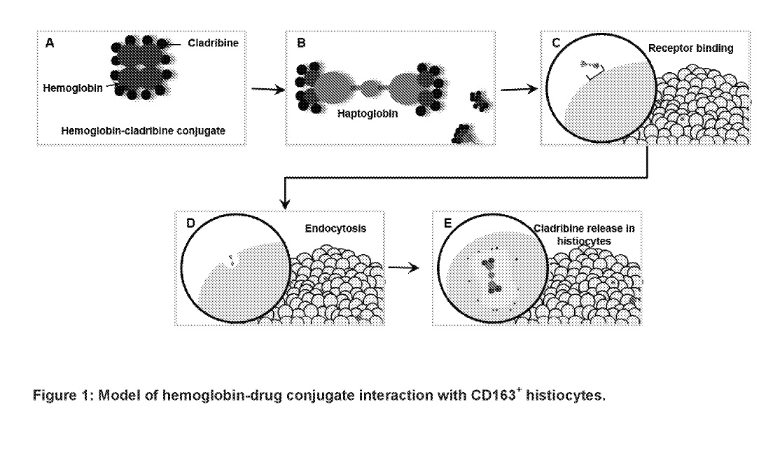

FIG. 1 is a schematic showing hemoglobin-drug conjugate interaction with CD163⁺ histiocytes. As shown in FIG. 1, administration of hemoglobin-cladribine (anti-proliferative) and hemoglobin-ribavirin (immunomodulatory) to CD163⁺ expressing histiocytes provides a unique opportunity to inhibit histiocyte expansion directly and also to reduce the inflammatory response through the reduced production of pro-inflammatory cellular mediators such as cytokines, interleukins and related molecules. After conjugation of multiple molecules of cladribine or ribavirin to one molecule of hemoglobin though means that do not interfere with haptoglobin binding and receptor interaction, the complex binds to haptoglobin (B) and is recognized by the scavenger receptor of hemoglobin-haptoglobin, CD163 (C). The complex is uptaken by histiocytes and a high drug load is released in the cytosol of the cell. Furthermore, conjugation of cladribine or ribavirin to hemoglobin avoids their distribution to non-target cells thus potentially allowing for an increase in potency through concentration in the target cells and a decrease in non-specific (i.e., systemic) toxicity through reduced exposure to non-target cells, through improved clearance to the target cells.

In another embodiment, the CD163 ligand is attached to two different drugs. In a specific embodiment, the CD163 ligand is hemoglobin which is attached to both cladribine and ribavirin.

In another embodiment, the CD163 ligand is an antibody or antibody fragment that binds CD163. Antibodies that bind to CD163 can be obtained commercially or can be prepared using techniques well known in the art. Antibodies that bind CD163 include, but are not limited to, EDHu-1 antibody from Novus Biologicals; the Mac2-158 or Mac2-48 antibodies from Trillium Diagnostics LLC; the EPR 14336 or EPR 14643 antibodies from Abcam; and the K20-T, 6E10.1G6, 6D3.2F3, 5C6-FA7, 215a27, 2B12, GH1/61, SP96 or 10D6 antibodies from Novus Biologicals.

Antibodies or antibody fragments can be coupled to drugs using techniques known in the art. For example, conjugation of drugs to anti-CD163 antibodies may be through endogenous or engineered cysteine, lysine or other residues or components of the antibody via succinimide ester, maleimide, iodoacetamide, caproic acid, hydrazone, or stable disulfide moieties, or other known methods in the art,

any combination of chemistry thereof.

In one embodiment, the ligand-drug conjugate is an anti-CD163 antibody-cladribine antibody-drug conjugate (ADC).

In another embodiment, the ligand-drug conjugate is an anti-CD163-ribavirin ADC.

The CD163 ligand-drug conjugates described herein may be formulated into pharmaceutical compositions for administration to subjects and/or use in subjects in a biologically compatible form suitable for administration in vivo. The CD163 ligand-drug conjugates can be prepared under cGMP conditions by using a process for either producing recombinant proteins or extracting native proteins from human sources with subsequent purification, characterization and functional analysis. The drug components may be conjugated to a protein carrier without altering the drug activity or the protein carrier itself. The compositions described herein can be prepared by per se known methods for the preparation of pharmaceutically acceptable compositions that can be administered to subjects, such that an effective quantity of the active substance is combined in a mixture with a pharmaceutically acceptable vehicle. Suitable vehicles are described, for example, in Remington's Pharmaceutical Sciences (Remington's Pharmaceutical Sciences, 20^{th} ed., Mack Publishing Company, Easton, Pa., USA, 2000). On this basis, the compositions include, albeit not exclusively, solutions of the substances in association with one or more pharmaceutically acceptable vehicles or diluents, and contained in buffered solutions with a suitable pH and iso-osmotic with the physiological fluids.

Pharmaceutical compositions include, without limitation, lyophilized powders or aqueous or non-aqueous sterile injectable solutions or suspensions, which may further contain antioxidants, buffers, bacteriostats and solutes that render the compositions substantially compatible with the tissues or the blood of an intended recipient. Other components that may be present in such compositions include water, surfactants (such as Tween), alcohols, polyols, glycerin and vegetable oils, for example. Extemporaneous injection solutions and suspensions may be prepared from sterile powders, granules, tablets, or concentrated solutions or suspensions. Proteins may be supplied, for example but not by way of limitation, as a lyophilized powder which is reconstituted with sterile water or saline prior to administration to the patient.

Pharmaceutical compositions may comprise a pharmaceutically acceptable carrier. Suitable pharmaceutically acceptable carriers include essentially chemically inert and nontoxic compositions that do not interfere with the effectiveness of the biological activity of the pharmaceutical composition. Examples of suitable pharmaceutical carriers include, but are not limited to, water, saline solutions, glycerol solutions, ethanol, N-(1(2,3-dioleyloxy)propyl)N,N,N-trimethylammonium chloride (DOTMA), diolesylphosphotidyl-ethanolamine (DOPE), and liposomes. Such compositions should contain a therapeutically effective amount of the compound, together with a suitable amount of carrier so as to provide the form for direct administration to the patient.

The compositions may be in the form of a pharmaceutically acceptable salt which includes, without limitation, those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylarnino ethanol, histidine, procaine, etc.

The CD163 ligand-drug conjugates and/or pharmaceutical compositions described herein may be administered to, or used in, living organisms including humans, and animals. The term “subject” or “animal” as used herein refers to any member of the animal kingdom, preferably a mammal, more preferably a human being.

Accordingly, the present application provides a pharmaceutical composition for treating CD163⁺ histiocytosis comprising an effective amount of a CD163 ligand-drug conjugate and a carrier. In one embodiment, the pharmaceutical composition comprises a hemoglobin-drug conjugate such as a hemoglobin-cladribine conjugate. In another embodiment, the pharmaceutical composition comprises an anti-CD163 antibody-drug conjugate such as an anti-CD163 antibody-cladribine conjugate. In another embodiment, the pharmaceutical composition comprises a hemoglobin-drug conjugate such as a hemoglobin-ribavirin conjugate. In another embodiment, the pharmaceutical composition comprises an anti-CD163 antibody-drug conjugate such as an anti-CD163 antibody-ribavirin conjugate.

Administration of an “effective amount” of the CD163 ligand-drug conjugates and/or pharmaceutical compositions is defined as an amount effective, at dosages and for periods of time necessary to achieve the desired result. For example, an effective amount of a substance may vary according to factors such as the disease state, age, sex, and weight of the individual, and the ability of the recombinant protein to elicit a desired response in the individual. Dosage regime may be adjusted to provide the optimum therapeutic response. For example, several divided doses may be administered daily or the dose may be proportionally reduced as indicated by the exigencies of the therapeutic situation.

As used herein, and as well understood in the art, “to treat” or “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e., not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “a cell” includes a single cell as well as a plurality or population of cells. Administering a ligand-drug conjugate to a cell includes both in vitro and in vivo administrations.

The following non-limiting examples are illustrative of the present invention:

### EXAMPLES

### Example 1: Characterization of Cell Models of Histiocytosis by Analyzing expression of CD163, CD68, CD1a and s100 by flow cytometry

To analyze the characteristic markers of histiocytes (positive for CD163 and CD68, and negative for CD1a and s100) flow cytometry experiments were performed with histiocytic lymphoma-derived cell lines SU-DHL-1 (macrophage-like), U937 (monocyte-like) and with U937 cells treated with phorbol 12-myristate 13-acetate and dexamethasone (PMA/DEX), which induces differentiation into a macrophage-like cell type. Approximately 50% of SU-DHL-1 and U937 (treated with PMA/DEX) and 3% of U937 cells expressed CD163. The cell lines did not express CD68, CD1a and only a small population of U937 cells (with or without PMA/DEX treatment) expressed s100.

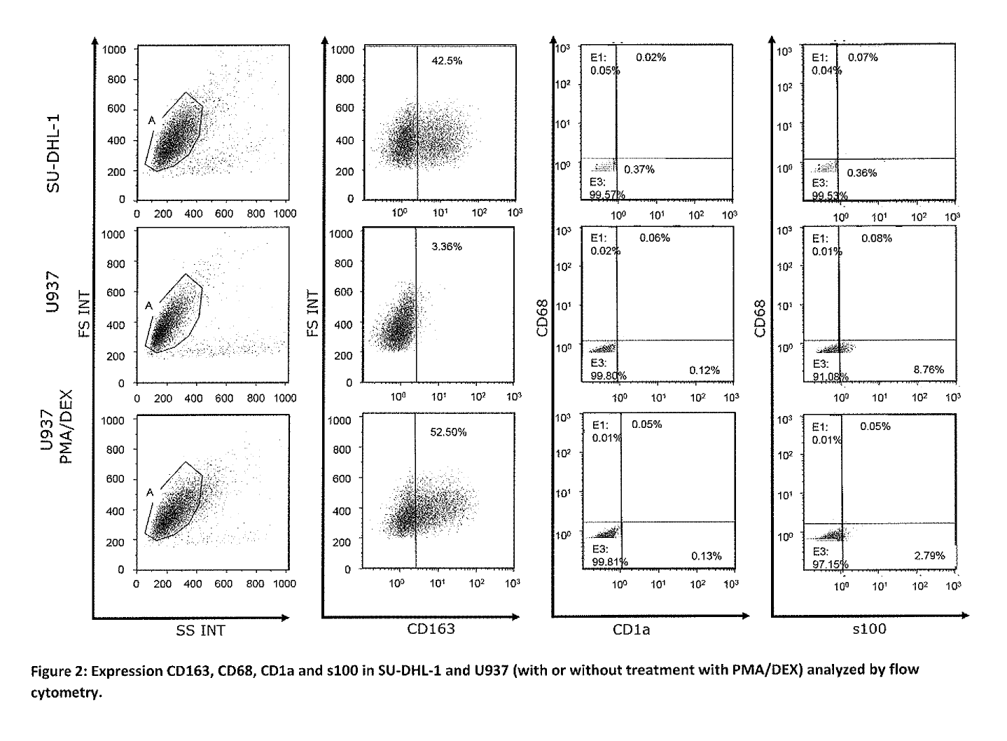

The results are shown in FIG. 2. Cells were stained with anti-CD163-APC, anti-CD68-PE, anti-CD1a-FITC, and anti-s100-FITC antibody and flow cytometry was performed on gated cell population. Isotype controls were used to set the gates for CD163, CD68, CD1a and s100 negative cells. Approximately 50% of SU-DHL-1 and U937 PMA/DEX cells (U937 cells treated with PMA and DEX) and 3% of U937 cells express CD163. None of the cell lines expresses CD68 or CD1a. U937 cells with or without treatment with PMA/DEX express low levels of s100. U937 cells were isolated from pleural effusion of a patient with histiocytic lymphoma and have the morphology of monocytes. SU-DHL-1 cells were isolated from peritoneal effusion of a patient with diffuse histiocytic lymphoma and have a lymphoblast-like morphology (ATCC). Due to the variable expression of CD163 and lack of CD1a, s100 and CD68 the cell lines can be considered as histiocytes-like cells with monocyte and macrophage-like morphology.

### Example 2: Potency of Hemoglobin-Cladribine Tested on an in Vitro Model of Histiocytosis

Histiocytic lymphoma-derived SU-DHL-1 and U937 (with or without PMA/DEX treatment) cell lines were treated with hemoglobin-cladribine, hemoglobin, cladribine or okadaic acid (induces apoptosis and functions as a positive control). The administration of hemoglobin-cladribine or cladribine alone, but not hemoglobin alone, resulted in dose-dependent growth inhibition of SU-DHL-1 and U937 cells. The average half maximal inhibitory concentration (IC₅₀) of hemoglobin-cladribine and cladribine was 0.626 μM and 0.204 μM for SU-DHL-1 (n=3) and 0.256 μM and 0.042 μM for U937 (n=3) cells, respectively. The difference between the hemoglobin-cladribine conjugate and cladribine 1050s were not statistically significant within each cell line. The IC₅₀for U937 cells treated with PMA/DEX could not be determined (ND), as no activity was measured. Hemoglobin-cladribine was demonstrated to be as potent as the free drug.

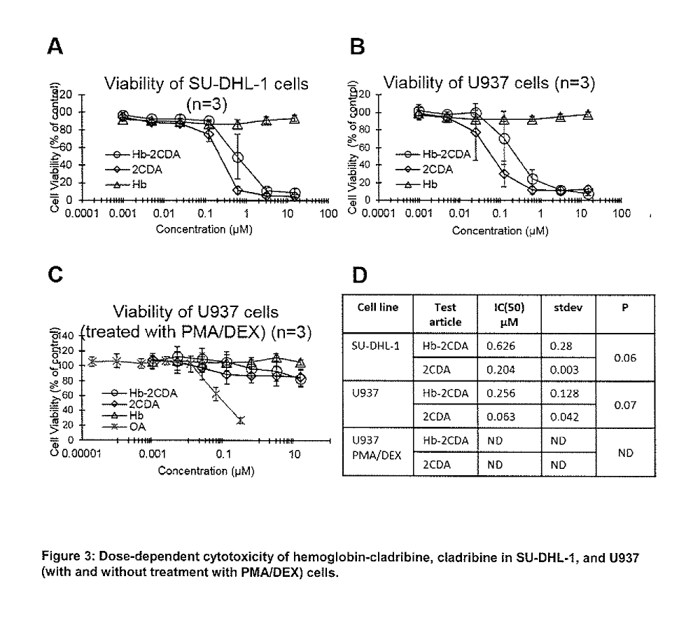

The results are shown in FIG. 3. Cell lines SU-DHL-1 (A), U937 (B) and U937 treated with PMA/DEX (C) were treated with hemoglobin, hemoglobin-cladribine, cladribine or okadaic acid for 48 hours and cell viability measured by mitochondrial activity-based PrestoBlue® assay in three independent experiments. The mean half-maximal inhibitory concentration (IC₅₀) of hemoglobin-cladribine and cladribine were determined and shown to be similar (D). PMA: phorbol 12-myristate 13-acetate; DEX: Dexamethasone; Hb-2CDA: hemoglobin-cladribine conjugate; 2CDA: cladribine; Hb: hemoglobin; OA; Okadaic acid.

### Example 3: Apoptosis-Inducinq Effect of Hemoqlobin-Cladribine in an in Vitro Model of Histiocytosis

SU-DHL-1 and U937 cell lines and PMA/DEX treated U937 cells were treated with hemoglobin-cladribine or cladribine alone. CD163 expression and indicators of early and late apoptosis (annexin V and 7AAD, respectively) were measured by flow cytometry. The administration of hemoglobin-cladribine or cladribine resulted in over 80% of the cells showing indicators of late apoptosis (annexin V positive and 7AAD positive). After-treatment with cladribine 27% of the CD163 positive cells were found to be in early apoptosis (annexin V positive and 7AAD negative), compared to 42% with hemoglobin-cladribine treatment, indicating that CD163+ proliferating cells are more sensitive to the cytotoxic effect of the hemoglobin-cladribine conjugate.

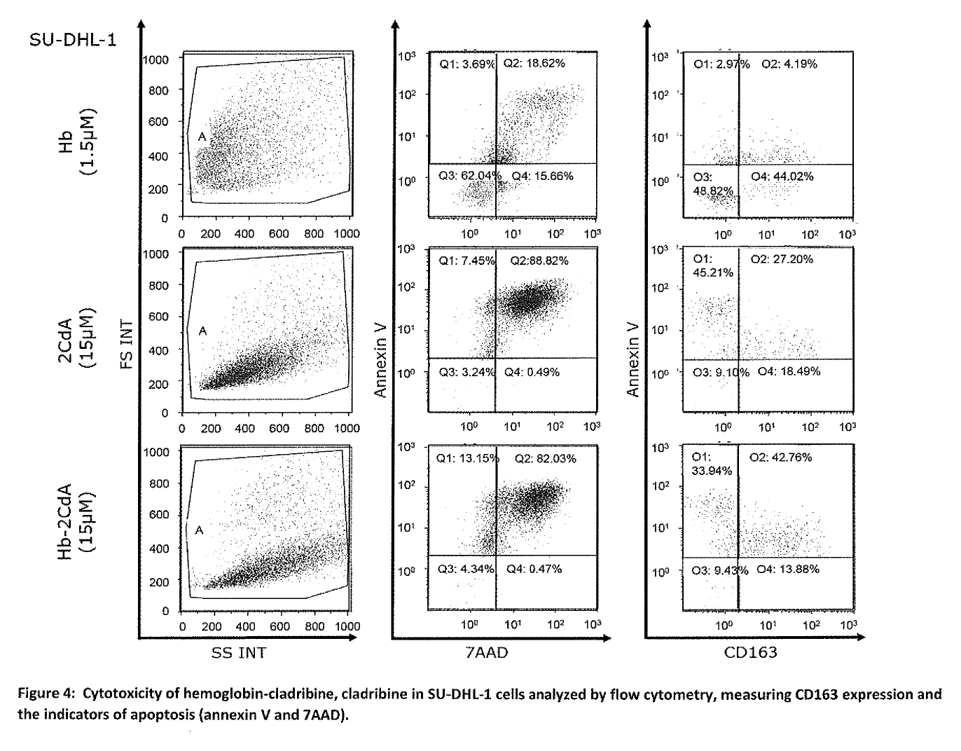

The results are shown in FIG. 4. SU-DHL-1 cells treated for 48 hours with hemoglobin, cladribine or hemoglobin-cladribine were stained with anti-CD163-PE antibody (Mac2-158), annexin V and 7AAD. Treatment of cells with cladribine or hemoglobin-cladribine resulted in 88% or 82% of cells being in late apoptosis, respectively (Annexin V positive and 7AAD positive). Twenty-seven or 42% of CD163 positive cells were found to be in early apoptosis (annexin V positive, 7AAD negative) after cladribine or hemoglobin-cladribine treatment, respectively.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

### LIST OF REFERENCES

1. Brookes S, Biessels P, Ng NF, Woods C, Bell DN, Adamson G. Bioconjug Chem. 2006 Mar-Apr;17(2):530-7. Synthesis and characterization of a hemoglobin-ribavirin conjugate for targeted drug delivery.
2. Dickson BC, Pethe V, Chung CT, Howarth DJ, Bilbao JM, Fornasier VL, Streutker CJ, Sugar LM, Bapat B. Virchows Arch. 2008 Feb;452(2):221-7. Epub 2008 Jan 10. Systemic Erdheim-Chester disease.
3. Dagna L. Girlanda S, Langheim S, Rizzo N, Bozzoko EP, Sabbadini MG and Ferrarini M. Rheumatology. 2010;49:1203-1206. Erdheim-Chester disease: report on a case and new insights on its immunopathogenesis.
4. Gong L, He XL, Li YH, Ren KX, Zhang L, Liu XY, Han XJ, Yao L, Zhu SJ,

Lan M, Zhang W. Pathol Res Pract. 2009;205(9):601-7. Epub 2009 Apr 1. Clonal status and clinicopathological feature of Erdheim-Chester disease.
5. Kristiansen M, Graversen JH, Jacobsen C, Sonne 0, Hoffman HJ, Law SK, Moestrup SK. Nature. 2001 Jan 11;409(6817):198-201. Identification of the haemoglobin scavenger receptor.
6. Law, SK, Micklem, KJ, Shaw, JM, Zhang, XP, Dong, Y, Willis, AC, and Mason, DY, A new macrophage differentiation antigen which is a member of the scavenger receptor superfamily. (1993). Eur. J. lmmunol. 23(9): 2320-2325.
7. Levy GA, Adamson G, Phillips MJ, Scrocchi LA, Fung L, Biessels P, Ng NF, Ghanekar A, Rowe A, Ma MX, Levy A, Koscik C, He W, Gorczynski R, Brookes S, Woods C, McGilvray ID, Bell D. Targeted Delivery of Ribavirin Improves Outcome of Murine Viral Fulminant Hepatitis via Enhanced Anti-Viral Activity. Hepatology, Vol. 43, No. 3, 2006.
8. Murakami Y, Wataya-Kaneda M, Terao M, Azukizawa H, Murota H, Nakata Y, Katayama I. Case Rep Dermatol. 2011 May;3(2):107-12. Epub 2011 May 17. Peculiar distribution of tumorous xanthomas in an adult case of erdheim-chester disease complicated by atopic dermatitis.
8. Purgina B, Jaffe R, Monaco SE, Khalbuss WE, Beasley HS, Dunn JA, Pantanowitz L. Cytojournal. 2011;8:22. Epub 2011 Dec 27. Cytomorphology of Erdheim-Chester disease presenting as a retroperitoneal soft tissue lesion.
10. Soota K and Maliakkal B. World J Gastroenterol. 2014 20(43)16184-16190. Ribavirin induced hemolysis: A novel mechanism of action against chronic hepatitis C virus infection.
11. Stoppacciaro A, Ferrarini M, Salmaggi C, Colarossi C, Praderio L, Tresoldi M, Beretta AA and Sabbadini MG. Arthritis & Rheumatism. 2006;54(12):4018-4022. Immunohistochemical evidence of a cytokine and chemokine network in three patients with Erdheim-Chester disease.
12. Van den Heuvel MM, Tensen CP, van As JH, Van den Berg TK, Fluitsma DM, Dijkstra CD, Döpp EA, Droste A, Van Gaalen FA, Sorg C, Högger P, Beelen RH. J Leukoc Biol. 1999 Nov;66(5):858-66. Regulation of CD 163 on human macrophages: cross-linking of CD163 induces signaling and activation.

We claim:

1. A method of treating CD163⁺ histiocytosis comprising administering an effective amount of a CD163 ligand-drug conjugate to an animal or cell in need thereof.

2. The method of claim 1, wherein the CD163⁺ histiocytosis is a non-Langerhans form of histiocytosis.

3. The method of claim 1, wherein the CD163⁺ histiocytosis is Erdheim-Chester disease.

4. The method of claim 1, wherein the drug is an anti-proliferative drug.

5. The method of claim 1, wherein the drug is an immunomodulatory drug.

6. The method of claim 4, wherein the anti-proliferative drug is cladribine.

7. The method of claim 5, wherein the immunomodulatory drug is ribavirin.

8. The method of claim 1, wherein the CD163 ligand is hemoglobin.

9. The method of claim 1, wherein the CD163 ligand is hemoglobin and the drug is cladribine.

10. The method of claim 1, wherein the CD163 ligand is hemoglobin and the drug is ribavirin.

11. The method of claim 1 comprising use of two different CD163 ligand-drug conjugates.

12. The method of claim 11 wherein one CD163 ligand-drug conjugate is hemogloblin-cladribine and the second CD163 ligand-drug conjugate is hemoglobin-ribavirin.

13. The method of claim 1, wherein the CD163 ligand is an anti-CD163 antibody.

14. The method of claim 1, wherein the animal is a human.

15. The method of claim 1 wherein the CD163 ligand is conjugated to two different drugs.

16. The method of claim 15 wherein one drug is cladribine and the second drug is ribavirin.

17. A pharmaceutical composition for treating CD163⁺ histiocytosis comprising an effective amount of a CD163 ligand-drug conjugate and a carrier.

18. The pharmaceutical composition of claim 17 wherein the CD163 ligand is hemoglobin.

19. The pharmaceutical composition of claim 18 wherein the drug is cladribine.

20. The pharmaceutical composition of claim 18 wherein the drug is ribavirin.

21. The pharmaceutical composition of claim 17 wherein the CD163 ligand is an antibody that binds CD163 or a fragment thereof.

22. The pharmaceutical composition of claim 21 wherein the drug is cladribine.
